(19) United States
(12) Patent Application Publication
(10) Pub. No.: US 2008/0269734 A1
(43) Pub. Date: Oct. 30, 2008

(54) Optical Array for Treating Biological Tissue

(76) Inventors: Agustina Vila Echague, Milton, MA (US); Jayant D. Bhawalkar, Brighton, MA (US); James C. Hsia, Weston, MA (US); Paul R. Lucchese, Sudbury, MA (US); Yacov Domankevitz, Newton, MA (US)

Correspondence Address: PROSKAUER ROSE LLP, ONE INTERNATIONAL PLACE, BOSTON, MA, 02110, US

(21) Appl. No.: 11/757,028

(22) Filed: Jun. 1, 2007

Related U.S. Application Data
(63) Continuation-in-part of application No. 11/796,146, filed on Apr. 26, 2007.

(51) Int. Cl. A61B 18/18

(52) U.S. Cl. 606/15

(57) ABSTRACT

Skin can be treated by penetrating an epidermis of the skin with a plurality of waveguides. Each waveguide has an end, which is positioned within a dermis of the skin. Electromagnetic radiation can be delivered through the plurality of waveguides to the dermis having a port wine stain for a time sufficient to selectively destroy a cutaneous blood vessel within the port wine stain. The time is less than a thermal diffusion time between the epidermis and the dermis to prevent forming substantial unwanted thermal injury within the epidermis.

### CROSS-REFERENCE TO RELATED APPLICATION

This application is a continuation in part of U.S. patent application Ser. No. 11/796,146 filed Apr. 26, 2007, which is owned by the assignee of the instant application and the disclosure of which is incorporated herein by reference in its entirety.

### FIELD OF THE INVENTION

The invention relates generally to apparatus and methods for treating biological tissue using electromagnetic radiation. In particular, the invention relates to an optical array for treating biological tissue.

### BACKGROUND OF THE INVENTION

A port wine stain (PWS) is a congenital, progressive, vascular malformation of the dermis involving capillaries and possibly perivenular nerves. Port wine stains occur in approximately three out of one thousand live births. Although a PWS may be found anywhere on the body, they mostly appear on the face and are noted over the dermatome distribution of the first and second trigeminal nerves.

In early childhood, PWS are faint pink macules, but the lesions tend to darken progressively to red-purple and by middle age, often become raised as a result of the development of vascular papules or nodules and occasionally tumors. The hypertrophy of underlying bone and soft tissue occurs in approximately two-thirds of the patients with PWS, and serves to further disfigure the facial features of many children.

Prior art treatments for PWS include scalpel surgery, ionizing radiation, skin grafting, dermabrasion, cryosurgery, tattooing, electrotherapy and flashlamp-pumped pulsed dye lasers. Light passing through the epidermis is preferentially absorbed by hemoglobin which is the major chromophore in blood in the ectatic capillaries in the upper dermis. The radiant energy is converted to heat causing thermal damage and thrombosis in the targeted vessels. Some studies have shown that the flashlamp-pumped pulsed dye laser produce good results in many pediatric and adult patients. However, laser treatments of PWS face the challenge that the overlying epidermal pigment layer comprises a barrier or an optical shield through which the light must first pass to reach the underlying PWS blood vessels. The absorption of laser energy by melanin causes localized heating in the epidermis and reduces the light dosage reaching the blood vessels, thereby decreasing the amount of heat produced in the targeted port wine stains and leading to suboptimal blanching of the lesion and/or unwanted thermal injury to the epidermis.

### SUMMARY OF THE INVENTION

The invention, in various embodiments, provides methods and apparatus for treating biological tissue. The biological tissue can be, but is not limited to, skin and hypodermal features such as port wine stains. The methods and apparatus can be for skin rejuvination. Apparatuses can include an array of needles to penetrate the biological tissue and fiber optics to deliver electromagnetic radiation to a subsurface volume of the biological tissue to treat the biological tissue. Advantages include effective and uniform treatment of deeper or selected layers of biological tissue without nonspecific damage to the upper or nonselected layers.

By applying electromagnetic radiation to subcutaneous tissue through a minimally invasive array of needles, the epidermis and the dermis can be spared from injury by the electromagnetic radiation. Furthermore, the electromagnetic radiation can diffuse within the subcutaneous tissue to effect a homogeneous treatment. Lower powers can also be used because the electromagnetic radiation is delivered directly to the targeted tissue and does not need to travel through the epidermis and/or dermis to reach the targeted tissue. At least a portion of the subcutaneous tissue can be treated for a PWS, and/or fibrosis and/or tightening of the skin can result without scarring the epidermis and/or dermis. Additionally, a portion of tissue can be suctioned or otherwise removed to facilitate treatment and/or mitigate the side effects of treatment.

In one aspect, the invention features a method for treating skin. The method includes penetrating an epidermis of the skin with a plurality of waveguides, each waveguide having an end. The method also includes positioning each end within a dermis of the skin, the dermis having a port wine stain. Additionally, the method includes delivering electromagnetic radiation through the plurality of waveguides to the dermis having the port wine stain for a time sufficient to selectively destroy a cutaneous blood vessel within the port wine stain. The time is less than a thermal diffusion time between the epidermis and the dermis to prevent forming substantial unwanted thermal injury within the epidermis.

In another aspect, the invention features a method for treating skin. The method includes penetrating a surface of a target region of the skin with a plurality of waveguides, each waveguide having an end. The method also includes positioning each end within the target region of the skin. Additionally, the method includes delivering electromagnetic radiation through the plurality of waveguides to the target region of skin to affect (i) at least one pigmentary abnormality disposed in an epidermal region of the target region and (ii) at least one vascular abnormality disposed in a dermal region of the target region.

In still another aspect, the invention features an apparatus for treating skin. The apparatus includes a first plurality of waveguides, each first waveguide having a first end, the first plurality of waveguides adapted for penetrating an epidermis of the skin, positioning each first end at about a first depth within the skin, and delivering electromagnetic radiation through the first plurality of waveguides to form a plurality of first injuries about the first depth. The apparatus also includes a second plurality of waveguides, each second waveguide having a second end, the second plurality of waveguides adapted for penetrating the epidermis, positioning each second end at about a second depth within the skin, and delivering electromagnetic radiation through the second plurality of waveguides to form a plurality of second injuries about the second depth.

In yet another aspect, the invention features a method for treating skin. The method includes penetrating an epidermis of the skin with a first plurality of waveguides, each first waveguide having a first end, and a second plurality of waveguides, each second waveguide having a second end. The method also includes positioning each first end at about a first depth within the skin and each second end at about a second depth within the skin. Additionally, the method includes delivering electromagnetic radiation through the first plurality of waveguides to form a plurality of first injuries about the first depth and delivering electromagnetic radiation through the second plurality of waveguides to form a plurality of second injuries about the second depth.

In still yet another aspect, the invention features a method for treating skin. The method includes penetrating an epidermis of the skin with a plurality of waveguides, each waveguide having an end. The method also includes positioning each end at about a first depth within the skin and delivering electromagnetic radiation through the plurality of waveguides to form a plurality of first injuries about the first depth. Additionally, the method includes positioning each end at about a second depth within the skin and delivering electromagnetic radiation through the second plurality of waveguides to form a plurality of second injuries about the second depth.

In other examples, any of the aspects above, or any apparatus or method described herein, can include one or more of the following features.

In various embodiments, the methods include delivering the electromagnetic radiation substantially simultaneously to multiple depths within the dermis. The methods can include delivering the electromagnetic radiation while the plurality of waveguides are being positioned within the dermis to treat multiple depths within the dermis. The methods can also include delivering electromagnetic radiation while the plurality of waveguides are being removed from the dermis to treat multiple depths within the dermis.

In some embodiments, the methods include positioning each end at multiple depths within the dermis of the skin and delivering electromagnetic radiation through the plurality of waveguides to the multiple depths within the dermis, to treat multiple layers or strata of the port wine stain. The methods can include delivering the electromagnetic radiation substantially simultaneously to the at least one pigmentary abnormality and the at least one vascular abnormality. The methods can also include cooling a surface of the epidermal region of the target region of skin to prevent substantial unwanted injury to at least a portion of the epidermal region.

In certain embodiments, the methods include positioning each end within the target region of the skin at a first depth to treat the at least one vascular abnormality, and repositioning each end within the target region of the skin at a second depth to treat the at least one pigmentary abnormality.

In various embodiments, the plurality of first injuries or the plurality of second injuries comprise a volume of necrotic thermal injury. The plurality of first injuries or the plurality of second injuries can partially denature collagen to cause the skin to rejuvenate. The plurality of first injuries or the plurality of second injuries can accelerate collagen synthesis in the skin to cause the skin to rejuvenate. The plurality of first injuries or the plurality of second injuries can elicit a healing response that produces substantially unwrinkled skin. The plurality of first injuries or the plurality of second injuries can activate fibroblasts which deposit increased amounts of extracellular matrix constituents in the skin.

In some embodiments, the plurality of first injuries or the plurality of second injuries are intervened by substantially undamaged skin. The methods can include forming a plurality of noncontiguous second injuries, disposed relative to the plurality of first injuries, to form a pattern of interspersed first injuries and second injuries. The plurality of first injuries can be shallower than the plurality of second injuries.

In certain embodiments, the electromagnetic radiation delivered to the first depth and the electromagnetic radiation delivered to the second depth differ in at least one parameter. The parameter can include at least one of fluence, wavelength, or pulse duration.

Other aspects and advantages of the invention will become apparent from the following drawings and description, all of which illustrate principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIGS. 1A-1C illustrate an exemplary apparatus having a base member and a plurality of needles and fiber optics for treating biological tissue.

FIGS. 2A-2C illustrate exemplary fiber optic tips.

FIGS. 3A-3B illustrate an exemplary needle with a fiber optic and a vacuum.

FIG. 4 illustrates an exemplary apparatus having a base member and a plurality of needles for treating biological tissue.

FIGS. 5A-5B illustrate exemplary fiber optic systems.

FIG. 6 illustrates the anatomy of a port wine stain.

FIGS. 7A-7C illustrate a method for treating biological tissue.

FIG. 8 illustrates another method for treating biological tissue.

FIG. 9 illustrates still another method for treating biological tissue.

FIG. 10 illustrates yet another method for treating biological tissue.

FIGS. 11A-11B show an exemplary region of treated skin.

FIGS. 12A-12B show another exemplary region of treated skin.

FIGS. 13A-13C illustrate an exemplary apparatus having a base member and a plurality of waveguides for treating biological tissue.

FIG. 14 shows an absorbent pad including an absorbent material.

FIGS. 15A-15B show a region of skin treated with a puncturing device.

FIGS. 16A-16B show a puncturing device with an absorbent pad.

### DETAILED DESCRIPTION OF THE INVENTION

A plurality of waveguides formed in an array pattern can be inserted into biological tissue. The waveguides can be positioned in the tissue so that a subsurface volume of the biological tissue can be treated. Electromagnetic radiation is delivered using the waveguides to treat the subsurface volume.

In certain embodiments, a treatment can be for one or more of the following indications: acne, erythema, fat, cellulite, oily skin, pigmented lesions, pores, scarring, vascular lesions (including port wine stains), pigmented lesions, and wrinkles, as well as for skin rejuvenation, hair removal, and hair regrowth. Target chromophores can include water, fat, collagen, blood or a blood component, melanin, or other commonly targeted skin chromophores in cosmetic and dermatologic treatments. Vascular lesions, such as PWS, telangiectasia and hemangiomas, are characterized by abnormally enlarged blood vessels. Pigmented lesions are non-vascular disfigurements of the skin caused by an abnormally high concentration of melanin in localized areas of the skin. Such pigmented lesions include freckles, age or liver spots, cafe au lait birthmarks, lentigines, nevi, melanomes, nevus of Ota and lentigo maligna.

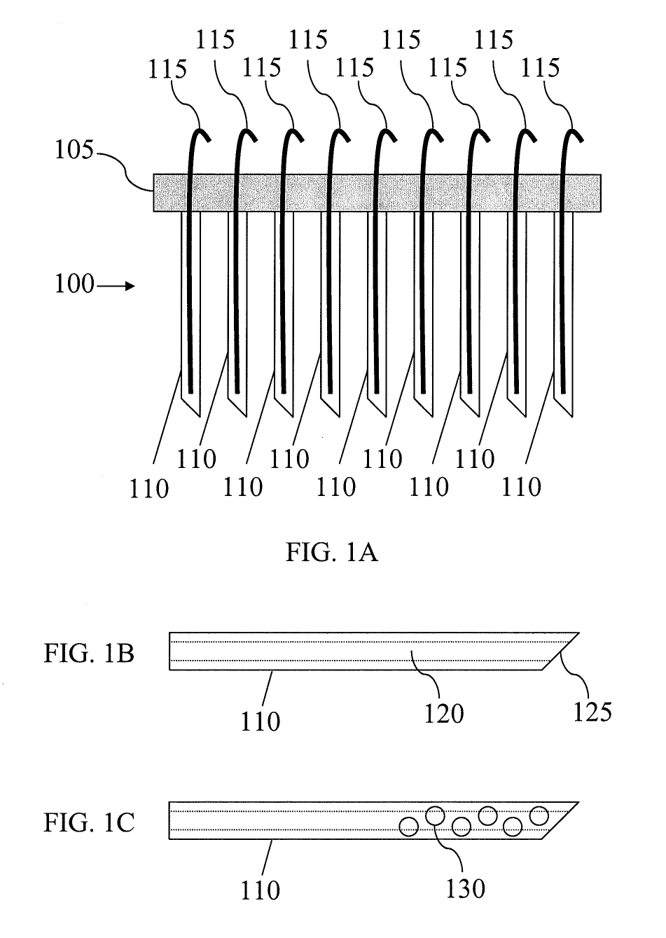

FIG. 1A illustrates an apparatus 100 for treating biological tissue including a base member 105, a plurality of needles 110 extending from the base member 105, and a plurality of fiber optics 115. The base member 105 can be made from a metal, plastic, or polymer material. The plurality of needles 110 can be attached to the base member 105, or can be removable. The base member 105 can be flexible, which can allow the plurality of needles 110 extending from the base member 105 to match a contour of the biological tissue.

FIG. 1B illustrates a needle 110 in detail. The needle 110 defines a bore 120 capable of receiving a fiber optic 115 and has an end 125.

FIG. 1C illustrates another embodiment of a needle 110 in detail. The needle 110 can define one or more openings 130 that allow electromagnetic radiation to radiate from the needle 110 from a region other than about the end 125. The one or more openings 130 can facilitate simultaneous treatment at more than one depth within the biological tissue.

In various embodiments, each needle 110 is adapted for penetrating biological tissue to a depth of about 0.5 mm to about 30 mm from a surface of the biological tissue. A needle 110 can be adapted to penetrate biological tissue to a depth of about 0.5 cm to about 2 cm. In certain embodiments, a needle 110 can be adapted to penetrate biological tissue to a depth of up to about 1 cm or about 2 cm. Indications such as PWS can extend even deeper within the skin and, in different embodiments, the needle 110 can be adapted to penetrate the biological tissue to any depth necessary to treat the indication. The diameter of each needle can be between about 0.2 mm and about 2 mm. In one embodiment, the diameter of each needle 110 is less than about 1 millimeter. In various embodiments, each needle 110 can be a different diameter and/or length. This can result in each needle 110 being positioned at a different depth within the subsurface volume of biological tissue, and can facilitate treatment at more than one depth. Variations in needle 110 length can also facilitate simultaneously treatment of a larger volume of biological tissue. Each needle 110 can be disposable. The base member 105 can be disposable. In one embodiment, the base member 105 and plurality of needles 110 can be a disposable, and/or can be a cartridge. Alternatively, the plurality of needles 110 and/or base member 105 can be sterilized and reusable. Each needle 110 can include stainless steel or aluminum, and can be a 30 G needle or a 27 G needle. In one embodiment, the needle 110 can be a STERIJECT® Rimos or Mesoram needle, which can be used as a multiinjector for mesotherapy.

The plurality of needles 110 form an array capable of penetrating a biological tissue and positioning each end 125 within a subsurface volume of the biological tissue. The base member 105 can function as a depth gauge by limiting the depth to which a needle 110 can be inserted into the biological tissue. The base member 105 and the needle 110 can be adjustable, so that the length of the needle 110 extending from the base member 105 can be adjusted. In one embodiment, the array of needles 110 are passed through holes in a rigid frame or a base member 105 and epoxied or fused to the frame or a base member 105. A biocompatible epoxy or low temperature glass frit can be used to epoxy or fuse the needles 110. Each fiber optic 115 is adapted for insertion into the bore 120 of each needle 110, and each fiber optic 115 is capable of delivering electromagnetic radiation to the subsurface volume of the biological tissue to treat the biological tissue.

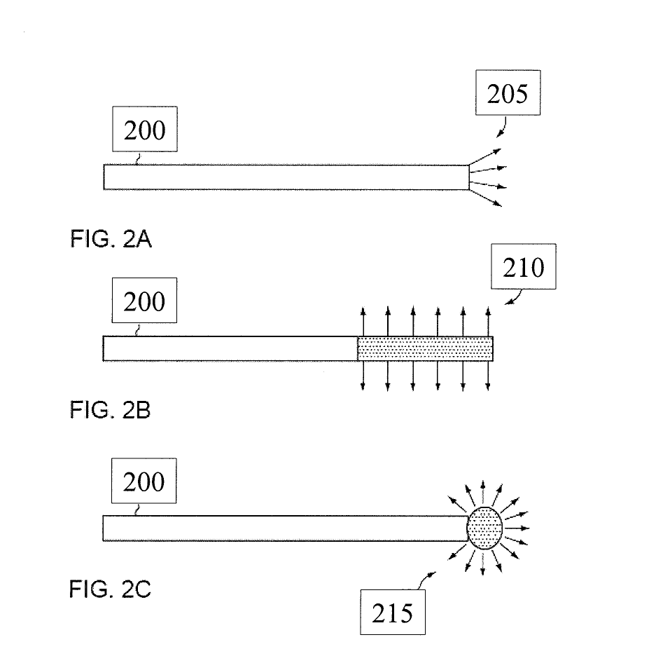

FIGS. 2A-2C illustrate exemplary fiber optic tips. In various embodiments, non-diffusing fiber optic tips can direct electromagnetic radiation substantially along the longitudinal axis of the fiber optic 200 to deliver the light to the biological tissue. In other embodiments, diffusing fiber tips can be used to deliver electromagnetic radiation to the biological tissue. Using diffusing fiber optic tips, electromagnetic radiation can be directed laterally from an end portion of the fiber optic 200, which can allow more precise heating and injury of the biological tissue and provide a more uniform and predictable treatment of the biological tissue. Furthermore, means known in the art can also be used to manipulate the end portion of the fiber optic 200. For example, the fiber optic 200 can be attached to a guide that can be manually or mechanically manipulated. The fiber optic can be adapted to be movable within the bore, extendable beyond the end of the needle, and/or retractable into the bore. The fiber optic can include sapphire. For example, the fiber optic or fiber optic tip can be sapphire.

FIG. 2A illustrates a fiber optic 200 with a bare fiber tip 205. The bare fiber tip 205 can be the simplest and least expensive design, and can be obtained by cleaving a fiber optic. In one embodiment, the fiber optic has a diameter of about 300 microns. For example, the fiber optic 200 can be a fiber optic manufactured or sourced from SCHOTT North America, Inc., which can cover a broad spectral range. The fiber optic 200 can be of diameter about 30 μm, 50 μm, 70 μm, or a different custom diameter. The arrows approximate the general direction of the propagation of electromagnetic radiation from the bare fiber tip 205.

FIG. 2B illustrates a fiber optic 200 with a linear diffuser tip 210. The light from the diffuser tip 210 is delivered laterally from the fiber optic 200 to the biological tissue. FIG. 2C illustrates a fiber optic 200 with a spherical ball-type diffuser tip 215, which emits light radially from the fiber tip. The diffuser tips 210 and 215 can include a scattering material, such as a polymer cover or a ceramic cover. The scattering material can overcome the index of refraction matching properties of the fiber optic and the adjacent fluid or biological tissue. The diffuser tips 210 and 215 are more expensive than bare fiber tip 205, but may provide better control of the light delivered. In various embodiments, the diffuser tip 210 or 215 can be permanently or removably affixed to the fiber optic 200. The diffuser tips 210 or 215 can be affixed using an adhesive, a bonding agent, a joining compound, an epoxy, a clip, a thread, other suitable mechanical connection or attachment means, or some combination thereof.

In various embodiments, the invention can include additional features to facilitate treatment of the biological tissue. For example, the apparatus can include a means for suctioning at least a portion of the biological tissue. The means of suctioning can be a needle 110 and a vacuum, or can be a different type of needle or tube, to remove and/or drain at least a portion of the subsurface volume of biological tissue.

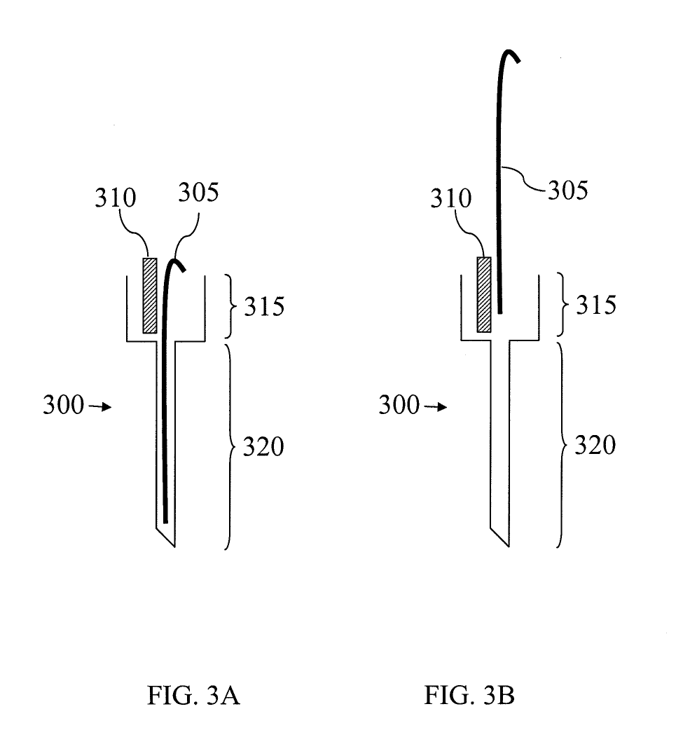

FIG. 3A-3B illustrate an exemplary needle 300 with a fiber optic 305 and a vacuum 310 for suctioning at least a portion of the biological tissue. The needle 300 can have two or more dimensions. For example, the needle 300 can have a first 315 portion of a first diameter positioned adjacent a base member, and a second 320 portion extending from the base member. The first 315 portion can affix the needle 300 to the base member, receive the fiber optic 305, and include the vacuum 310. The second 320 portion can penetrate a biological tissue, facilitate delivery of the fiber optic 305 to a subsurface volume of the biological tissue, and facilitate use of the vacuum 310 for suctioning at least a portion of the subsurface volume of biological tissue. The vacuum 310 can be used for suctioning tissue after it is melted and/or liquefied by electromagnetic radiation delivered by the fiber optic 305. FIG. 3B illustrates an arrangement where the fiber optic 305 is withdrawn from the second 320 portion before employing the vacuum 310 for suctioning tissue. Employing a needle with a fiber optic that can be retracted to allow suctioning can result in a needle with a smaller diameter.

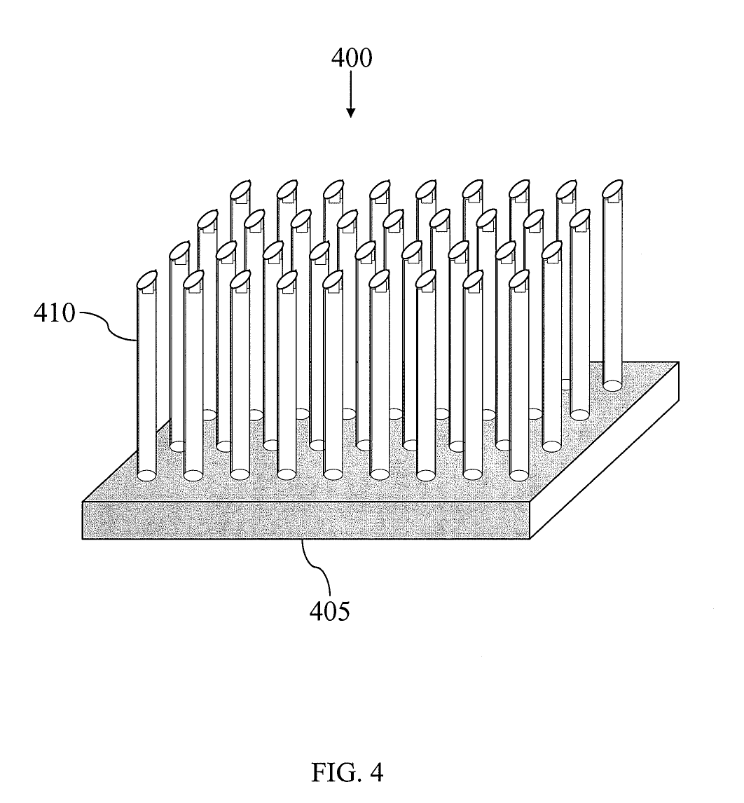

FIG. 4 illustrates another view of an apparatus 400 for treating biological tissue including a base member 405, a plurality of needles 410, and a plurality of fiber optics (not shown). The plurality of needles 410 can include the same features as the plurality of needles 110 described in FIG. 1. The apparatus 400 illustrates a regular, two dimensional array of the plurality of needles 410.

The invention is not limited to the number and/or arrangement of needles shown in FIGS. 1 and 4. For example, the invention includes apparatuses with regular and irregular, as well as one and two dimensional, arrays of two or more needles. Furthermore, the invention includes embodiments where the needle does not form a right angle with the base member. For example, the invention includes apparatuses where the plurality of needles forms an angle of about 45 degrees, or any other angle between about 30 degrees and about 90 degrees between the base member and each needle, to facilitate nonperpendicular entry into the biological tissue.

In various embodiments, the base member and/or needle array can have a diameter of about 10 cm, or dimensions up to about 10 by 10 cm, 5 by 5 cm, or 5 by 10 cm. The base member and/or needle array can be square, rectangular, circular, ovoid, or polygonal. Polygonal or other base members can be used for “tiling,” to cover a larger area by forming a regular pattern of individual treatment areas. In various embodiments individual needles can be spaced less than about 5 mm apart or between about 50 microns to about 2 mm apart. In some embodiments, individual needles can be spaced between about 500 microns to about 1 mm apart. In certain embodiments, needles are spaced about 0.5 mm or about 1 mm apart. The spacing between needles in an array need not be uniform, and can be closer in areas where a greater amount of damage or more precise control of damage in the target area of tissue is desired. In one embodiment, the array of needles can include pairs of needles separated from adjacent pairs by larger distances. Needles can be arranged in a regular or near-regular square, triangular, or other geometrical arrays. The pattern of damage and/or tissue reshaping can be controlled by adjusting the intensity and/or duration of power transmitted to individual fiber optics. An array of needles can distribute pressure over a larger area when puncturing the skin, to reduce pain and/or discomfort.

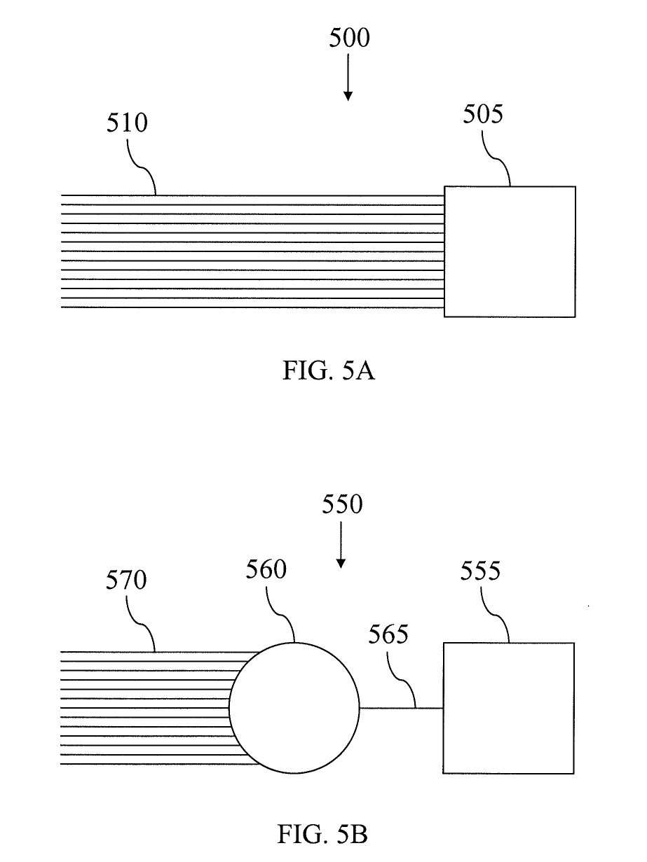

FIG. 5A illustrates an exemplary fiber optic system 500 including a source 505 of electromagnetic radiation and a plurality of fiber optics 510. The source 505 of electromagnetic radiation can be, for example, a plurality of individual diode lasers, each coupled to an individual fiber optic 510.

FIG. 5B illustrates an exemplary fiber optic system 550 including a source 555 of electromagnetic radiation coupled to a coupler 560 through a connector 565. A plurality of fiber optics 570 are adapted to receive electromagnetic radiation from the source 555 through the coupler 560. The source 555 can include, for example, an individual diode laser, which forms a laser beam that is split by the coupler 560 to deliver approximately the same quality and quantity of electromagnetic radiation to each individual fiber optic 570.

The invention is not limited to the number and/or arrangement of fiber optics shown in FIGS. 5A-5B. Rather, a fiber optic system can be adapted for virtually any number and arrangement of fiber optics and/or needles. A fiber optic system can also be adapted for fiber optics of varying length. In various embodiments, the plurality of fiber optics receives a beam of radiation from a source of electromagnetic radiation. The apparatus can include a source of electromagnetic radiation. The source of electromagnetic radiation can be a laser, a light emitting diode, an incandescent lamp, a flash lamp, or a gas discharge lamp. Each fiber optic can employ free-space coupling to deliver electromagnetic radiation to treat the biological tissue. The beam of electromagnetic radiation can have a power between about 0.1 watts and about 500 watts. In one embodiment, the power delivered by each fiber optic is less than about 1 W. The beam of electromagnetic radiation can have a pulse duration between about 0.1 microseconds and about 10 seconds.

A fiber optic system can include a control system that can control the fiber optics individually. In one embodiment, the control system can deliver electromagnetic radiation to a subset of the fiber optics. The subset of fiber optics can match a pattern of a target, to treat the target and spare surrounding tissue. For example, the target can be a vein and the controller can deliver electromagnetic radiation to curvilinear array of fiber optics to treat the vein and to spare the tissue surrounding the vein. The control system can control the properties of electromagnetic radiation delivered to each fiber optic. For example, the fluence, wavelength, and/or duration of the electromagnetic radiation delivered to each fiber optic can be controlled.

In various embodiments, tissue in the target region can be heated to a temperature of between about 50° C. and about 100° C., although higher and lower temperatures can be used depending on the application. In one embodiment, the temperature is between about 55° C. and about 70° C. In one embodiment, the temperature is between about 70° C. and about 100° C.

In various embodiments, the beam of electromagnetic radiation can have a wavelength between about 400 nanometers and about 10,600 nanometers. The beam of radiation can have a wavelength between about 330 and about 600 nm, about 585 nm and about 600 nm, or between about 700 and about 800 nm. In some embodiments, the beam of radiation has a wavelength of about 500 nm, 532 nm, 585 nm, 595 nm, 755 nm, 780 nm, 1210 nm, or 1310 nm. The source of the beam of radiation can be an alexandrite laser, a variable pulsed duration alexandrite laser, a Nd:Yag laser, a diode laser, or a flashlamp pumped pulsed dye laser. The beam of radiation can have a wavelength that is absorbed by endogenous cutanious chromophores including hemoglobin, melanin, and/or other chromophores within the PWS or lesion.

In various embodiments, the beam of radiation can have a fluence up to about 500 J/cm². In one embodiment, the beam of radiation has a fluence of between about 60 J/cm² and about 300 J/cm², although higher and lower fluences can be used depending on the application. In one embodiment, the beam of radiation has a fluence between about 1 and 10 J/cm² and preferably between 2 and 4 J/cm². In another embodiment, the beam of radiation has a fluence of between about 60 J/cm² and about 150 J/cm². In one embodiment, the beam of radiation has a fluence between about 80 J/cm² and about 100 J/cm². High fluences can lead to better collagen shrinkage in a blood vessel wall and/or perivascular, and therefore better stenosis. Lower fluences can be appropriate in many embodiments because direct delivery of light to about the region of skin to be treated through a waveguide, as apposed to transmission through the epidermis and/or dermis, can mitigate the fluence necessary to effect treatment.

In various embodiments, the beam of radiation can have a pulse duration between about 10 ms and about 300 ms, although a longer and shorter pulse duration can be used depending on the application. In one embodiment, the beam of radiation has a pulse duration between about 20 ms and about 100 ms. In one embodiment, the beam of radiation has a pulse duration between about 20 ms and about 60 ms. In one embodiment, the beam of radiation has a pulse duration between about 20 ms and about 40 ms. In one embodiment, the beam of radiation has a pulse duration between about 40 ms and about 60 ms. In one embodiment, the beam of radiation has a pulse duration of about 40 ms. In one embodiment, the beam of radiation has a pulse duration greater than about 40 ms. In one embodiment, the beam of radiation has a pulse duration of less than 1 μs, and preferably less than 500 ns.

In various embodiments, the beam of radiation can be delivered at a rate of between about 0.1 pulse per second and about 10 pulses per second, although faster and slower pulse rates can be used depending on the application.

In various embodiments, the parameters of the radiation can be selected to deliver the beam of radiation to a predetermined depth. In some embodiments, the beam of radiation can be delivered to the target area up to about 10 mm below a surface of the skin although shallower or deeper depths can be selected depending on the application. In some embodiments, the beam of radiation can be delivered to the target area up to about 5 mm below a surface of the skin. In some embodiments, the beam of radiation can be delivered to the target area up to about 4 mm below a surface of the skin. In some embodiments, the beam of radiation can be delivered to the target area up to about 2 mm below a surface of the skin. In some embodiments, the beam of radiation can be delivered to the target area up to about 1 mm below a surface of the skin.

A cooling system can be used to modulate the temperature in a region of biological tissue and/or minimize unwanted thermal injury to untargeted region of biological tissue. For example, the system can cool the skin before, during, or after delivery of radiation, or a combination of the aforementioned. Cooling can include contact conduction cooling, evaporative spray cooling, convective air flow cooling, or a combination of the aforementioned. In one embodiment, the handpiece includes a skin contacting portion that can be brought into contact with a region of skin. The base member can be cooled. A cooling plate can also be cooled. The cooling pale can be adjacent the base member. The cooling pale can define a plurality of holes through which the needles can pass. By cooling only a region of the target region or by cooling different regions of the target region to different extents, the degree of thermal injury of regions of the target region can be controlled.

U.S. patent application Ser. No. 11/645,222 and U.S. Pat. Nos. 5,312,395, 5,599,342, and 5,814,040, the disclosures of which are incorporated by reference herein in their entirety, disclose treatment parameters and features that can be advantageously employed with the invention.

In various embodiments, local anesthesia can be administered to the patient. Anesthesia can be delivered prior to and/or during delivering the beam of radiation or penetrating the biological tissue. In one embodiment, the anesthesia can be injected directly into the biological tissue. Anesthesia delivery can also include applying a topical anesthetic to the biological tissue. Alternatively, the method can include the use of general anesthesia. Performing the procedure without anesthesia can be beneficial for patients who may have an adverse reaction to anesthesia. Use of local anesthetic can also reduce cost of a procedure by eliminating the need for an anesthesiologist.

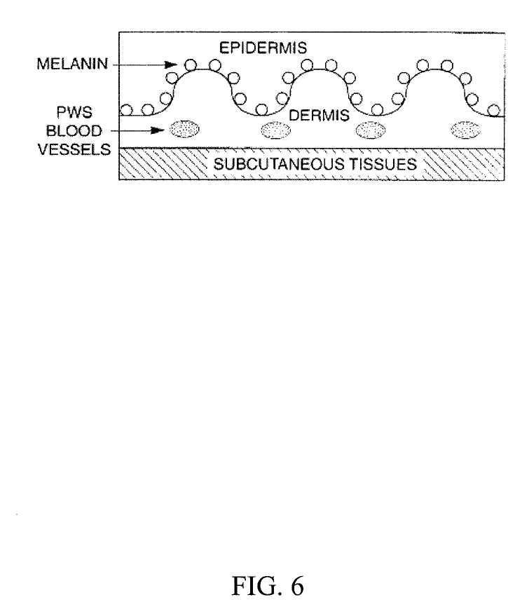

FIG. 6 illustrates the anatomy of a port wine stain. Histopathological studies of PWS show a normal epidermis overlying an abnormal plexus of dilated blood vessels located on a layer in the dermis. Endogenous chromophores including water, collagen, fat, melanin, and hemoglobin can absorb the electromagnetic radiation intended to treat the biological tissue. Therefore, in treatments that transmit electromagnetic radiation through the epidermis to the PWS, the overlying epidermal layer can be a barrier or an optical shield through which the light must first pass to reach the underlying PWS blood vessels. The absorption of laser energy by endogenous chromophores can cause localized heating in the epidermis and reduces the light dosage reaching the blood vessels, thereby decreasing the amount of heat produced in the targeted port wine stains and leading to suboptimal blanching of the lesion and/or unwanted thermal injury to the epidermis.

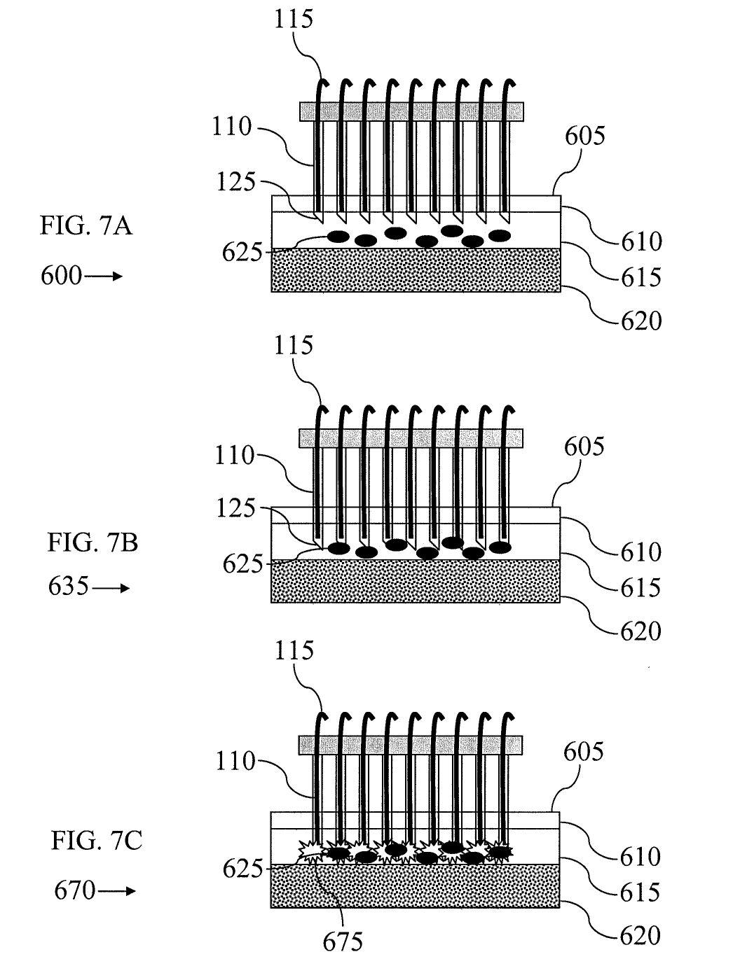

FIGS. 7A-7C illustrate a method for treating biological tissue. The biological tissue can be skin having a surface 605, an epidermis 610, a dermis 615, and subcutaneous tissue 620. The dermis 615 can include PWS blood vessels 625.

In FIG. 7A, step 600 shows the plurality of needles 110 penetrating the surface 605 of the biological tissue. The plurality of needles 110 also penetrates the epidermis 610 and the dermis 615. Penetrating the surface 605 of the biological tissue with the plurality of needles 110 can form an angle of about 45 degrees between the surface 605 of the biological tissue and each needle. In various embodiments, penetrating the surface 605 of the biological tissue with the plurality of needles 110 forms an angle of about 30 degrees and about 90 degrees between the surface 605 of the biological tissue and each needle.

In FIG. 7B, step 635 shows the positioning of each end 125 within the dermis 615. In particular, each end 125 can be positioned substantially within and/or about the PWS blood vessels 625. In some embodiments, the plurality of fiber optics 115 are positioned within the plurality of needles 110 after step 635. In other embodiments, the fiber optics 115 are positioned within the plurality of needles 110 prior to step 600, in which case the fiber optics 115 may require adjustment after step 635. The fiber optics 115 can be positioned within the plurality of needles 110 by a push switch mechanism. The fiber optics 115 can be disposable.

In FIG. 7C, step 670 shows the delivery of electromagnetic radiation 675 through the plurality of fiber optics 115 to the dermis 615 to treat the biological tissue. In particular, the electromagnetic radiation can be delivered substantially within and/or about the PWS blood vessels 625, to induce thermal injury to the PWS and mitigate thermal injury to the surrounding tissue. Thermal injury can include at least one of denaturation, necrosis, blanching, photothermolysis, destruction, and irreversible destruction. The electromagnetic radiation 675 can be delivered to multiple depths within the dermis or biological tissue. For example, the method can treat multiple layers or strata of the PWS. The method can also affect at least one pigmentary abnormality disposed in an epidermal region of the target region and at least one vascular abnormality disposed in a dermal region of the target region. In various embodiments, the electromagnetic radiation 675 is delivered to multiple depths within the dermis or biological tissue while the plurality of needles 110 are being positioned within, and/or removed from, the biological tissue. The method illustrated in FIGS. 7A-7C is not limited to treating PWS and can be employed, in various embodiments and with various additions or modifications, for treating other conditions in biological tissue.

The electromagnetic radiation 675 can thermally injure at least a portion of the PWS blood vessels 625 and/or surrounding tissue. The method can include allowing the thermally injured PWS blood vessels and/or surrounding tissue to escape through the needle holes. The method can also include suctioning the thermally injured PWS blood vessels and/or surrounding tissue through the plurality of needles 110 and/or another means for suctioning. In some embodiments, the needle is partially retracted to expose at least a portion of the fiber optics 115 to the PWS blood vessels 625 and/or surrounding tissue. The electromagnetic radiation 675 can also be delivered through one or more openings defined by the needle 110.

In various embodiments, the method can include the additional steps of (i) removing each end 125 from the dermis 615; (ii) translating and/or rotating the plurality of needles 110 relative to the biological tissue; (iii) penetrating the surface 605 of the of biological tissue with the plurality of needles 310; (iv) positioning the each end 125 within a second subsurface volume (not shown) of the dermis 615; and (v) delivering electromagnetic radiation 675 through each fiber optic 115 inserted within the bore to the second subsurface volume of the biological tissue to treat the biological tissue. Translating or rotating the plurality of needles 110 relative to the biological tissue can form a larger area of coverage (e.g., positioning the each end 125 within a second subsurface volume) and/or higher coverage of a single area (e.g., repositioning each end 125 within a portion of the subsurface volume that was already treated).

In some embodiments, the method can include moving a portion of the at least one fiber optic within the subsurface volume of biological tissue while delivering electromagnetic radiation. For example, the plurality of needles 310 can be moved within the dermis 615 while delivering electromagnetic radiation 675 to maximize the amount of the PWS blood vessels 625 that are thermally injured. The melted and/or liquefied PWS blood vessels 625 can drain through the needle holes and/or be removed by suctioning. Suctioning can include removing the fiber optic 115 from at least a portion of the bore 120 and applying a vacuum 310. In various embodiments massage and/or irrigation can be employed to aid in the removal of melted and/or liquefied PWS blood vessels 625. In another example, blood within the PWS blood vessels 625 can be drained through the needle holes and/or be removed by suctioning prior to the delivery of the electromagnetic radiation 675. Drainage or removal of the blood can improve treatment of the PWS by at least one of facilitating collapse of the PWS blood vessels 625, reducing the volume of tissue to be thermally injured, and increasing the thermal injury to the PWS blood vessels 625 (e.g., more light is absorbed by the PWS blood vessels 625 in the absence or reduced presence of blood). In certain embodiments, the method can include mitigating pain and/or discomfort. For example, anesthesia can be administered before step 600 when the plurality of needles 110 penetrates the surface 605 of the biological tissue or after step 600. Anesthesia can also be administered during the treatment.

In various embodiments, the method can include cooling at least a portion of the biological tissue, to mitigate undesired thermal damage to the portion of the biological tissue. For example, the epidermis and/or dermis can be cooled in conjunction with delivering increased fluences of electromagnetic radiation to the subcutaneous tissue to mitigate undesired thermal damage to the epidermis and/or dermis while increasing the efficacy of treatment of the subcutaneous tissue. A member can apply pressure to and/or cool the skin, to displace blood from a region of biological tissue, to limit damage to blood vessels in the region of biological tissue.

In one embodiment, the method includes contacting the skin with a cooled plate to cool and numb the skin. The plate can define a plurality of holes. A plurality of needles 110 can penetrate the surface 605 of the biological tissue through the plurality of holes in the plate. Alternatively, the plate can be removed before the plurality of needles 110 penetrate the surface 605 of the biological tissue.

The treatment radiation can damage and/or destroy one or more PWS blood vessel cells so that at least a portion of the damaged cells can escape and/or can be drained from the treated region. At least a portion of the damaged cells can be carried away from the tissue through a biological process. In one embodiment, the body's lymphatic system can drain the damaged and/or destroyed cells from the treated region. In an embodiment where a cell is damaged, the cell can be viable after treatment. In one embodiment, a first portion of the fat cells is damaged and a second portion is destroyed. In one embodiment, a portion of the damaged and/or destroyed cells can be removed to selectively change the shape of the body region.

In some embodiments, the beam of radiation can be delivered to a target chromophore in the target region. Suitable target chromophores include, but are not limited to, water, collagen, fat, melanin, and hemoglobin. The energy absorbed by the chromophore can be transferred to the cell to damage or destroy the cell. For example, thermal energy absorbed by dermal tissue can be transferred to the PWS. In one embodiment, the beam of radiation is delivered to water within or in the vicinity of a PWS in the target region to thermally injure the PWS.

In various embodiments, treatment radiation can affect one or more cells and can cause sufficient thermal injury in the dermal region of the skin to elicit a healing response to cause the skin to remodel itself. This can result in more youthful looking skin. In one embodiment, sufficient thermal injury induces fibrosis of the dermal layer, fibrosis on a subcutaneous region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

In one embodiment, fatty tissue is heated by absorption of radiation, and heat can be conducted into dermal tissue proximate the fatty tissue. The fatty tissue can be disposed in the dermal tissue and/or can be disposed proximate to the dermal interface. A portion of the dermal tissue (e.g., collagen) can be partially denatured or can suffer another form of thermal injury, and the dermal tissue can be thickened and/or be strengthened as a result of the resulting healing process. In such an embodiment, a fat-selective wavelength of radiation can be used.

In one embodiment, collagen and/or water in the dermal tissue is heated by absorption of radiation. For example, in various embodiments, the radiation can have a wavelength of about 400 nm to about 2,600 nm, or about 1.3 microns to about 1.8 microns, which can target water and/or collagen absorption peaks. The dermal tissue can have disposed therein fatty tissue and/or can be overlying fatty tissue. A portion of the dermal tissue (e.g., collagen) can be partially denatured or can suffer another form of thermal injury, and the dermal tissue can be thickened and/or be strengthened as a result of the resulting healing process. A portion of the heat can be transferred to the fatty tissue, which can be affected. In one embodiment, water in the fatty tissue absorbs radiation directly and the tissue is affected by heat. In such embodiments, a water selective wavelength of radiation can be used.

In various embodiments, the invention can include photodynamic therapy (PDT). For example, a photosensitizer (e.g., aminolevulinic acid, ALA, or methyl aminolevulinate) can be administered to the subject, and the light can be delivered directly to the desired treatment site using a plurality of waveguides at virtually any location. Thus, the invention can include PDT treatments that are not limited by the transmission of light through the skin or biological tissue. The invention can also include treatments that deliver virtually any wavelength of light to activate the photosensitizer while reducing collateral damage. For example, longer wavelengths such as 630 nm are often used for PDT because shorter wavelengths are strongly absorbed by the melanin and cause collateral damage. However, shorter wavelengths can be more effective in activating photosensitizers like ALA. Thus the method can include a PDT treatment delivering blue light (e.g., about 400 nm) through a waveguide directly to a PWS, to increase photosensitizer activation and reduce collateral damage. In addition to PWS, the invention can include PDT for fatty tissue, cancerous tissue, and other tissue. For example, a lipid-soluble photosensitizer (e.g., hypericin, an extended quinone photosensitizer produced by St. John's wort) can be used to enhance the treatment, melting, removal, and thermal injury of fatty tissue. In other examples, the invention can include PDT for cancers including basal cell carcinoma and other skin cancers, sebaceous tumors, eccrine and apocrine tumors, lipomas, and generally any localized, protruding, or bulky tumor.

U.S. Pat. Nos. 5,810,801, 6,120,497, and 6,659,999 and U.S. patent application Ser. Nos. 10/241,273, 10/407,921, 10/698,970 and 11/148,051, the disclosures of which are incorporated by reference herein in their entirety, disclose treatment parameters and features that can be advantageously employed with the invention.

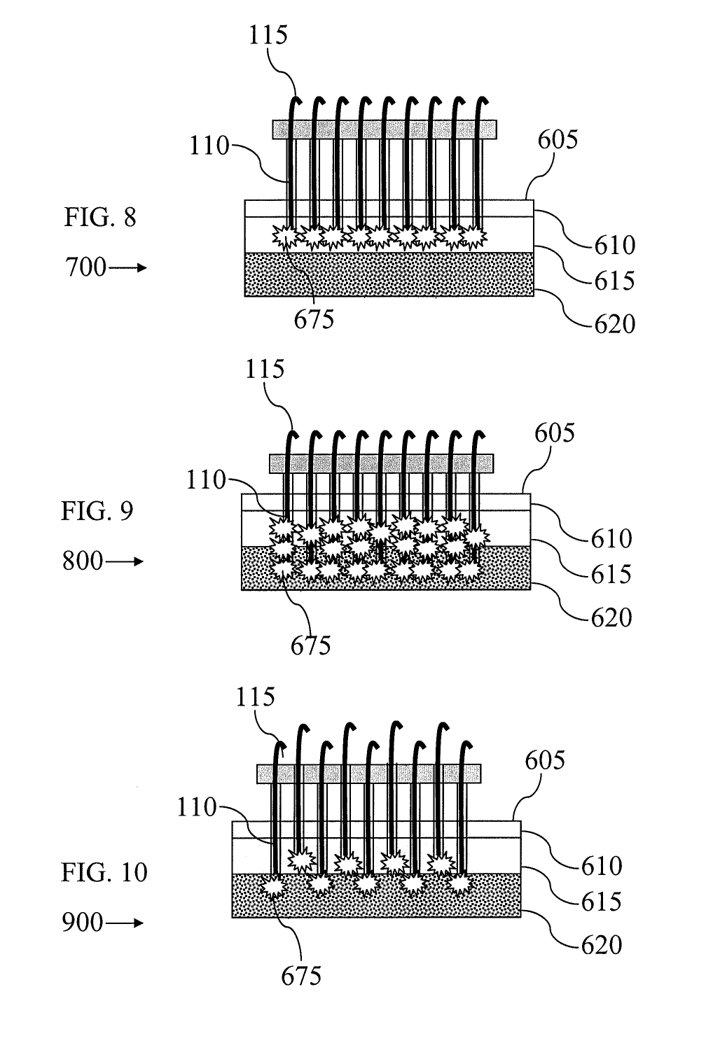

In FIG. 8, step 700 shows the delivery of electromagnetic radiation 675 through the plurality of fiber optics 115 to the dermis 615 to treat the biological tissue. The electromagnetic radiation 675 can denature collagen and/or otherwise injure at least a portion of the dermis 615. In various embodiments, step 700 can precede and/or follow step 670 shown in FIG. 7C. Step 700 can be a discreet step (e.g., position the plurality of fiber optics 115 within the dermis 615 and deliver electromagnetic radiation 675) or continuous (e.g., deliver electromagnetic radiation 675 while the fiber optics 115 are being inserted and/or withdrawn from the biological tissue). In various embodiments, a step like step 700 can include the delivery of electromagnetic radiation 675 through the plurality of fiber optics 115 to at least one of the surface 605, the epidermis 610, the dermis 615, and the subcutaneous tissue 620 to treat the biological tissue.

In FIG. 9, step 800 shows the simultaneous delivery of electromagnetic radiation 675 through the plurality of fiber optics 115 to the dermis 615 and to the subcutaneous tissue 620. The method of step 800 can be achieved by employing a needle 110 defining one or more openings that allow electromagnetic radiation to radiate from a region other than about the end like, for example, the needle 110 shown in FIG. 1C. The amount of electromagnetic radiation directed to a specific depth can be controlled by the number, size, and/or transmittance of the openings. The needle 110 can be positioned within the biological tissue and the intensity and/or duration of electromagnetic radiation directed to a specific depth can be controlled by the rate of insertion and/or withdrawal of the fiber optics 115.

In FIG. 10, step 900 shows the simultaneous delivery of electromagnetic radiation 675 through the plurality of fiber optics 115 to the dermis 615 and to the subcutaneous tissue 620. The method of step 900 can be achieved by employing a plurality of needles 110 of varying length. For example, one or more needles 110 can be within the dermis 615 and one or more needles 110 can be within the subcutaneous tissue 620. In other examples, needles 110 of varying length can be used for simultaneous delivery of electromagnetic radiation 675 to varying depths of the dermis 615 and/or subcutaneous tissue 620.

In various embodiments, a step like step 800 and/or 900 can include the simultaneous delivery of electromagnetic radiation 675 through the plurality of fiber optics 115 to at least two of the surface 605, the epidermis 610, the dermis 615, and the subcutaneous tissue 620 to treat the biological tissue. In some embodiments, a step like step 800 and/or 900 can include the delivery of electromagnetic radiation 675 through the plurality of fiber optics 115 to at least multiple depths within the surface 605, the epidermis 610, the dermis 615, and/or the subcutaneous tissue 620 to treat the biological tissue. In certain embodiments, such as step 800 and/or 900, the electromagnetic radiation is delivered approximately perpendicular to the axis of the needle 110. In one embodiment, the needles are spaced such that the electromagnetic radiation forms zones of thermal injury separated by substantially undamaged biological tissue.

The methods shown in FIGS. 7-10 can include the advantages of even heating of the biological tissue, delivery of electromagnetic radiation directly to the subsurface volume of biological tissue being targeted, and/or reducing trauma from the treatment. In various embodiments, the method can form a pattern of thermal injury within the biological tissue.

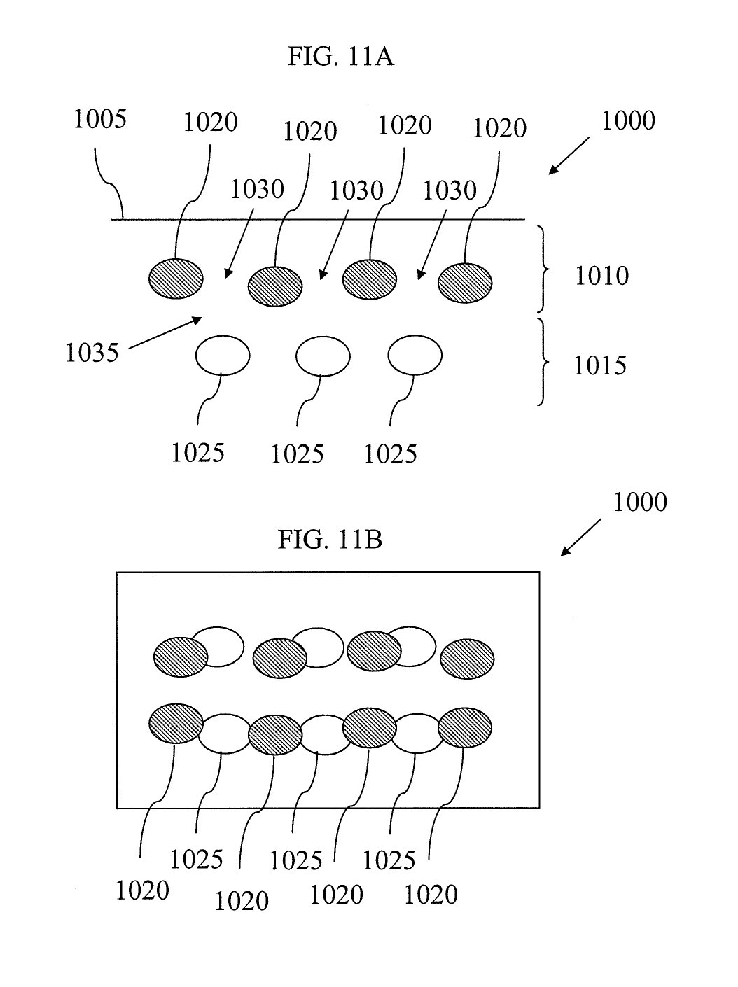

FIG. 11A shows a cross-section of an exemplary region of skin 1000 including a skin surface 1005, a first region 1010 of skin at a first depth, a second region 1015 of skin at a second depth, a plurality of first thermal injuries 1020 in the first region 1010, and a plurality of second thermal injuries 1025 in the second region 1015. Each plurality of thermal injuries can be separated by substantially undamaged skin 1030. The thermal injuries at the first depth can be separated from the thermal injuries at the second depth by an intermediate region of substantially undamaged skin 1035.

The first thermal injuries 1020 can be more severe than the second thermal injuries 1025. For example, the first thermal injuries 1020 can be necrotic thermal injuries within the epidermis, and the second thermal injuries 1025 can denature collagen within the dermis. Necrotic thermal injuries elicit a healing response from the skin. Denaturing collagen can accelerate collagen synthesis, tighten skin, mitigate wrinkles, and/or elicit a healing response. An interspersed plurality of first thermal injuries 1020 and second thermal injuries 1025 can intensify the skin's healing response and accelerate recovery and healing, as compared to a large, continuous thermal injury. Healing can initiate from less injured or substantially undamaged skin 1030 adjacent the plurality of first thermal injuries 1020 and/or second thermal injuries 1025.

FIG. 11B shows a top view of the region of skin 1000 shown in FIG. 11A. The first and second thermal injuries can form less than about 100% coverage of a target region of skin, which can be measured as the area corresponding to the thermal injuries as seen from the skin surface. In some embodiments, the first and second thermal injuries can form about 100% coverage of a target region of skin.

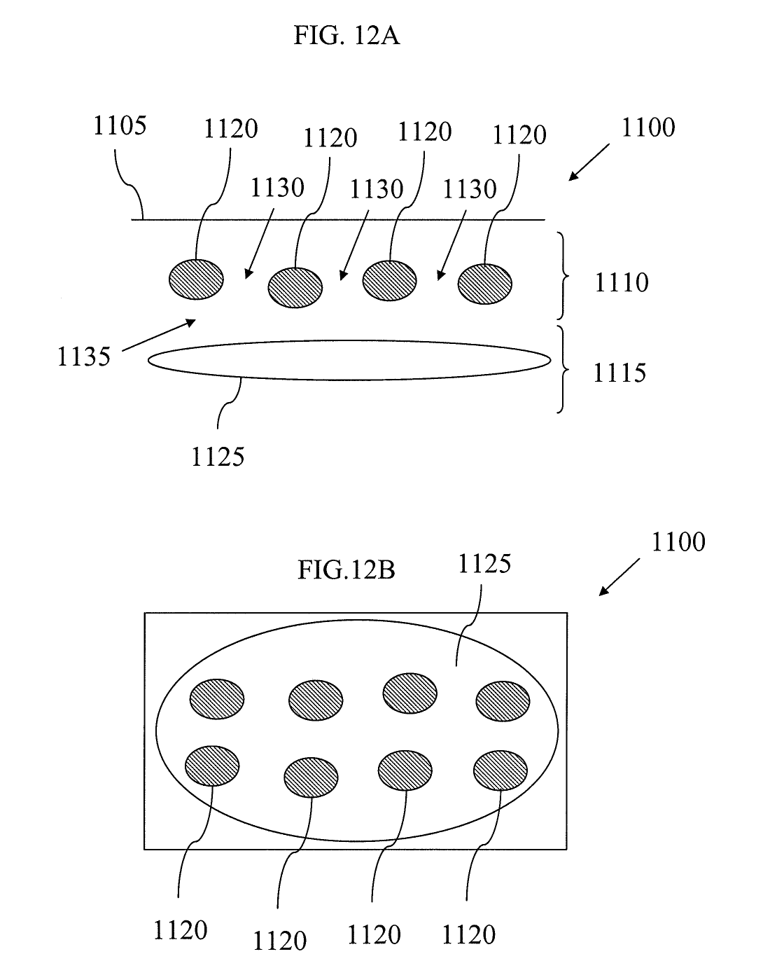

FIG. 12A shows a cross-section of an exemplary region of skin 1100 including a skin surface 1105, a first region 1110 of skin at a first depth, a second region 1115 of skin at a second depth, a plurality of first thermal injuries 1120 in the first region 1110, and a second thermal injury 1125 in the second region 1115. Each of the plurality of first thermal injuries 1120 can be separated by substantially undamaged skin 1130. The first thermal injuries 1120 at the first depth can be separated from the second thermal injury 1125 by an intermediate region of substantially undamaged skin 1135.

The first thermal injuries 1120 can be more severe than the second thermal injury 1125. For example, the first thermal injuries 1120 can be necrotic thermal injuries within the epidermis and the second thermal injury 1125 can denature collagen within the dermis. Necrotic thermal injuries elicit a healing response from the skin. Denaturing collagen can accelerate collagen synthesis, tighten skin, mitigate wrinkles, and/or elicit a healing response. The first thermal injuries 1120 overlying a second thermal injury 1125 can intensify the skin's healing response and accelerate recovery and healing, as compared to a large, continuous, severe thermal injury. Healing can initiate from less injured or substantially undamaged skin 1130 adjacent the plurality of first thermal injuries 1120 and/or second thermal injury 1125.

FIG. 12B shows a top view of the region of skin 1100 shown in FIG. 12A. The first and second thermal injuries can form about 100% coverage of a target region of skin, which can be measured as the area corresponding to the thermal injuries as seen from the skin surface. In some embodiments, the first and second thermal injuries can form less than about 100% coverage of a target region of skin.

In various embodiments, methods such as those illustrated in FIGS. 7A-10 can be employed to form patterns of thermal injury such as those shown in FIGS. 12A-12B, to rejuvenate skin.

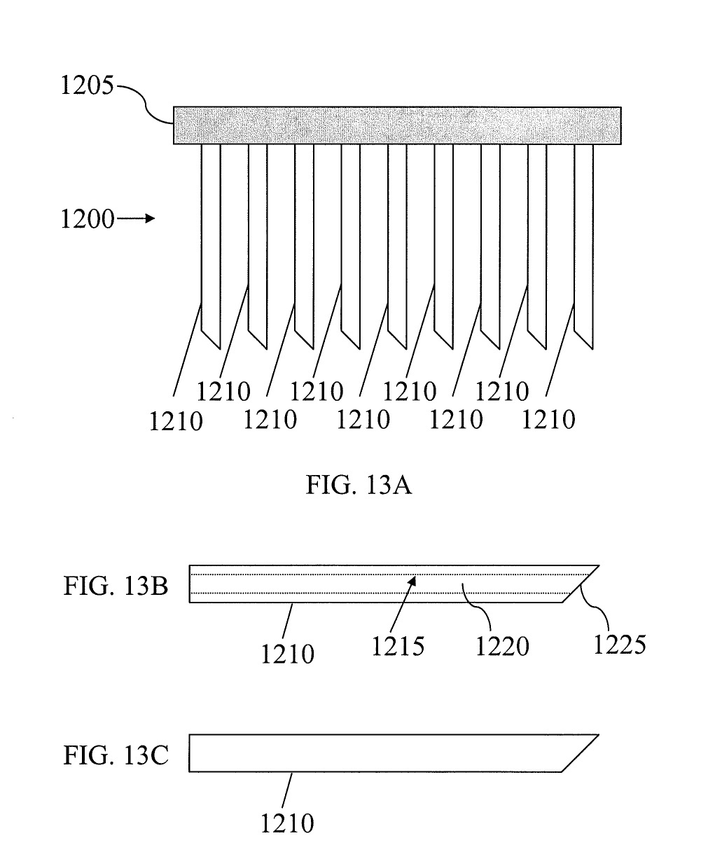

FIG. 13A illustrates an apparatus 1200 for treating biological tissue including a plurality of waveguides 1210 extending from a base member 1205. The base member 1205 can be made from a metal, plastic, or polymer material. The plurality of waveguides 1210 can be attached to the base member 1205, or can be removable. The base member 1205 can be flexible, which can allow the plurality of waveguides 1210 extending from the base member 1205 to match a contour of the biological tissue.

FIG. 13B illustrates one embodiment of a waveguide 1210 in detail. The waveguide 1210 can be a hollow waveguide. For example, the waveguide 1210 can be a needle that defines a bore 1220 and has an end 1225. In some embodiments, at least a portion of an inner surface 1215 has a coating to facilitate transmission of the electromagnetic radiation. The inner surface 1215 can be covered with a single or multilayer film, which for example, guides light by Bragg reflection (e.g., a photonic-crystal fiber). The film can be silver. Small prisms around the waveguide, which reflect light via total internal reflection, can be used. In other embodiments, the inner surface 1215 is not coated and can be polished metal. In various embodiments, the hollow waveguide 1210 can include silica, glass, sapphire, crystal, metal, and/or plastic materials. The hollow waveguide 1210 can be a naked waveguide or can be a waveguide inserted into the bore 120 of a needle 110. In various embodiments, the hollow waveguide 1210 can define one or more openings (not shown) that allow electromagnetic radiation to radiate from the waveguide 1210 from a region other than about the end 1225. The one or more openings can facilitate simultaneous treatment at more than one depth within the biological tissue.

FIG. 13C illustrates another embodiment of a waveguide 1210 in detail. The waveguide 1210 can be a solid waveguide including silica, glass, sapphire, crystal, metal, and/or plastic materials. The waveguide 1210 can include one or more layers and/or coatings to facilitate transmission of the electromagnetic radiation. In some embodiments, a rigid, solid waveguide 1210 is adapted to penetrate biological tissue. In other embodiments, a solid waveguide 1210 is inserted into the bore 120 of a needle 110. A waveguide can have similar dimensions to the needles and fiber optics described above. Each waveguide 1210 can be disposable. The base member 1205 can be disposable. In one embodiment, the base member 1205 and plurality of waveguides 1210 can be a disposable, and can be in the form of a cartridge. Alternatively, the waveguide 1210 and/or base member 1205 can be sterilized and reusable. The plurality of waveguides 1210 form an array capable of penetrating a biological tissue and positioning each end 1225 within a subsurface volume of the biological tissue. Each waveguide 1210 is capable of delivering electromagnetic radiation to the subsurface volume of the biological tissue to treat the biological tissue. A vacuum can be applied to the subsurface volume of biological tissue through a hollow waveguide 1210. Alternatively, the hollow and/or solid waveguide can be retracted from at least a portion of the bore 120 of a needle 110, and a vacuum can be applied to the subsurface volume of biological tissue through the needle 110. In some embodiments an apparatus can include one or more waveguides for delivering electromagnetic radiation, and one or more needles for applying a vacuum, to the subsurface volume of the biological tissue. In various embodiments, any of the needle and fiber optic features and methods described herein can be used with waveguides 1210.

In some embodiments, the biological tissue can be covered with an absorbent material to draw one or more fluids from the biological tissue. The absorbent material can be a wound dressing that includes a substance to draw fluid from the biological tissue to increase the biological tissue's response to the injury, remove unwanted or damaged biological tissue, and/or to induce shrinkage of the biological tissue.

Skin shrinkage can result in an improvement in the skin's appearance. For example, puncturing and treating the skin with radiation can damage or destroy selected tissue, and can elicit a healing response to cause the skin to remodel itself. Skin shrinkage can thicken the skin, tighten the skin, improve skin laxity, induce collagen formation, promote fibrosis of the dermal layer, and result in rejuvenation of the skin. In certain embodiments, improvement occurs in the dermal region of the skin. Furthermore, a treatment can include a series of treatment cycles, so that skin can be reduced gradually, and/or the skin can be tightened gradually, resulting in a more cosmetically appealing appearance.

The skin can shrink by a range of a factor of about 1 to about 10. In certain embodiments, the skin can shrink by at least a factor of about 1.25 to about 5. In some embodiments, the skin can shrink by at least a factor of about 1.1, 2, 3, or 4. Skin shrinkage can be measured by determining the percentage decrease in a volume of target tissue. Skin shrinkage can be measured by determining the percentage decrease in the surface area of the target tissue.

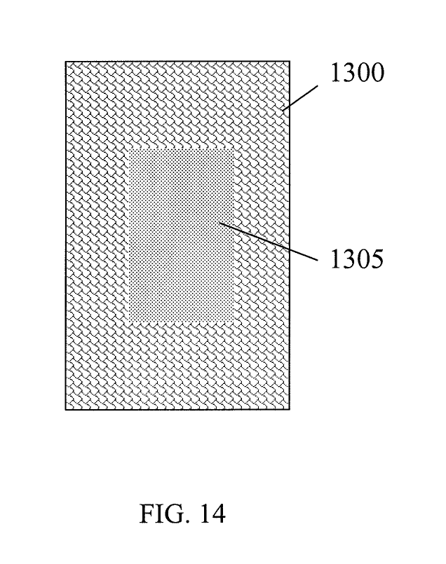

FIG. 14 shows an absorbent pad 1300 including an absorbent material 1305 disposed on the absorbent pad 1300. In certain embodiments, the absorbent pad 1300 alone is the absorbent material. A dressing can be applied to the target region of skin. The dressing can include the absorbent pad 1300 and the absorbent material 1305.

The absorbent material 1305 can draw fluid from the skin. The fluid can be one or more of a body fluid, a cellular fluid, damaged tissue, injured tissue, melted tissue, liquefied tissue, and water. The absorbent material 1305 can include a solid or a liquid. The absorbent material 1305 can include salt or glycerol. For example, the absorbent material 1305 can include at least one of a salt mixture or a composition including a salt. The absorbent material 1305 can be a desiccating agent, a solution adapted to draw a body fluid from the target region, or a solution adapted to draw a cellular fluid from the target region. The absorbent material can include an antiseptic, an antibiotic, and/or a disinfectant.

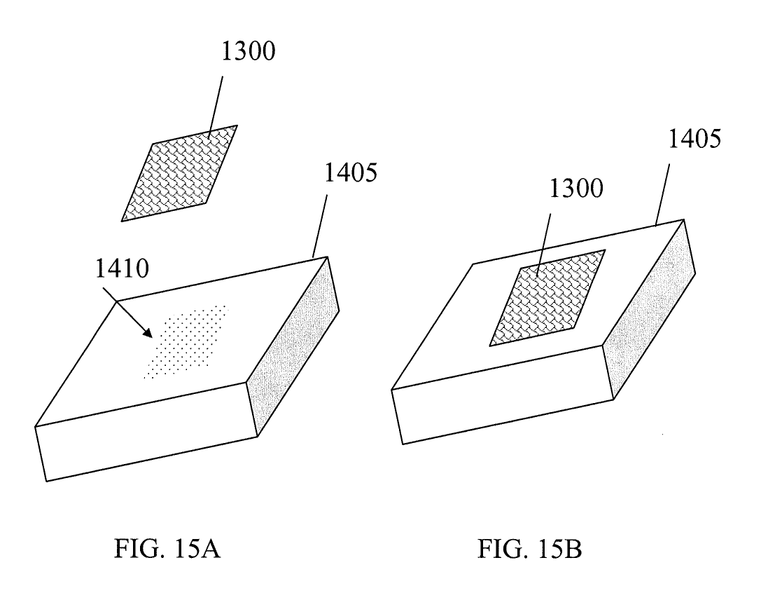

FIG. 15A shows a region of skin 1405 treated with a puncturing device to cause a plurality of puncture marks 1410. FIG. 15B shows the absorbent pad 1300 covering the region of skin 1405. In some embodiments, the absorbent pad 1300 or material 1305 is applied for a period of between about 1 minute and about 3 days. Depending on the treatment, longer and shorter time frames can be used. The absorbent pad 1300 or material 1305 can be applied for a period of at least 1 minute. In some embodiments, the absorbent pad 1300 or material 1305 can be applied for about 1 minute, about 15 minutes, about 30 minutes, about 60 minutes, about 2 hours, about 6 hours, about 12 hours, about 1 day, about 2 days, or about 3 days. In certain embodiments, a first pad can be removed from the skin and a second pad can be applied.

The absorbent pad 1300 or the absorbent material 1305 can cause the fluid to migrate from the target region of skin to the absorbent material 1305. For example, the fluid can migrate to an outer surface of the skin so the absorbent material 1305 can absorb the fluid.

The severity of the treatment can be varied, for example, by varying the density of skin punctures, the size of the needles, the depth of the punctures, and by varying the concentration of the topical agents used. More aggressive treatment may lead to beneficial skin shrinkage with a scar. Less aggressive treatments may produce beneficial skin shrinkage without producing a scar.

In certain embodiments, the absorbent material 1305 can be applied directly to the skin 1405. A bandage, e.g., the absorbent pad 1300, can be applied over the skin 1405 and the absorbent material 1305.

In certain embodiments, suction can be used to remove fluid from the biological tissue. For example, as the needles are removed from the biological tissue, the force of withdraw can draw fluid to the surface of the biological tissue. In some embodiment, a suction system or syringe is used.

In certain embodiments, the biological tissue can be irrigated after the biological tissue is punctured. This can include using a needle or syringe to inject a fluid into the biological tissue.

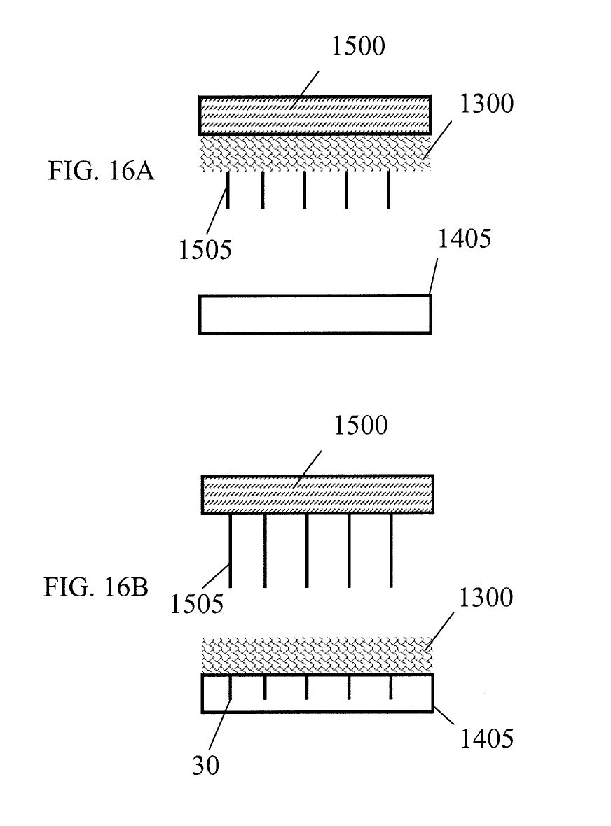

FIG. 16A shows an embodiment where the absorbent pad 1300 is affixed to a base member 1500. The base member 1500 is placed proximate to the skin 1405 so the needles 1505 can puncture the skin 1405. Referring to FIG. 16B, with base member 1500 withdrawn, the absorbent pad 1300 is ejected from the base member 1500 and the absorbent pad 1300 covers the skin, including the puncture marks 1510 remaining in the skin from the needles 1505. The absorbent pad 1300 can remove fluid from the skin 1405 to cause the skin to shrink 1405.

In certain embodiments, a beam of radiation can be applied through the surface of the biological tissue to affect the biological tissue. The beam of radiation can augment or complement the treatment using the waveguides or needles. The beam of radiation can be applied before, during, or after insertion of the waveguides or needles. For example, the beam of radiation can be delivered to the target region to thermally injure, damage, and/or destroy one or more fat cells. This can lead to reshaping of the biological tissue region as the skin size is reduced. The surface of the biological tissue can be cooled to protect overlying tissue.

In some embodiments, the beam of radiation can cause sufficient thermal injury in the dermal region of the skin to elicit a healing response to cause the skin to remodel itself. This can result in more youthful looking skin. In one embodiment, sufficient thermal injury induces fibrosis of the dermal layer, fibrosis on a subcutaneous fat region, or fibrosis in or proximate to the dermal interface. In one embodiment, the treatment radiation can partially denature collagen fibers in the target region. Partially denaturing collagen in the dermis can induce and/or accelerate collagen synthesis by fibroblasts. For example, causing selective thermal injury to the dermis can activate fibroblasts, which can deposit increased amounts of extracellular matrix constituents (e.g., collagen and glycosaminoglycans) that can, at least partially, rejuvenate the skin. The thermal injury caused by the radiation can be mild and only sufficient to elicit a healing response and cause the fibroblasts to produce new collagen. Excessive denaturation of collagen in the dermis causes prolonged edema, erythema, and potentially scarring. Inducing collagen formation in the target region can change and/or improve the appearance of the skin of the target region, as well as thicken the skin, tighten the skin, improve skin laxity, and/or reduce discoloration of the skin.

While the invention has been particularly shown and described with reference to specific embodiments, it should be understood by those skilled in the art that various changes in form and detail may be made without departing from the spirit and scope of the invention as defined by the appended claims.

1. A method for treating skin comprising:
penetrating an epidermis of the skin with a plurality of waveguides, each waveguide having an end;
positioning each end within a dermis of the skin, the dermis having a port wine stain; and
delivering electromagnetic radiation through the plurality of waveguides to the dermis having the port wine stain for a time sufficient to selectively destroy a cutaneous blood vessel within the port wine stain, the time less than a thermal diffusion time between the epidermis and the dermis to prevent forming substantial unwanted thermal injury within the epidermis.

2. The method of claim 1 further comprising delivering the electromagnetic radiation substantially simultaneously to multiple depths within the dermis.

3. The method of claim 1 further comprising delivering the electromagnetic radiation while the plurality of waveguides are being positioned within the dermis to treat multiple depths within the dermis.

4. The method of claim 1 further comprising delivering electromagnetic radiation while the plurality of waveguides are being removed from the dermis to treat multiple depths within the dermis.

5. The method of claim 1 further comprising:
positioning each end at multiple depths within the dermis of the skin; and
delivering electromagnetic radiation through the plurality of waveguides to the multiple depths within the dermis, to treat multiple layers or strata of the port wine stain.

6. A method for treating skin comprising:
penetrating a surface of a target region of the skin with a plurality of waveguides, each waveguide having an end;
positioning each end within the target region of the skin; and
delivering electromagnetic radiation through the plurality of waveguides to the target region of skin to affect (i) at least one pigmentary abnormality disposed in an epidermal region of the target region and (ii) at least one vascular abnormality disposed in a dermal region of the target region.

7. The method of claim 6 further comprising delivering the electromagnetic radiation substantially simultaneously to the at least one pigmentary abnormality and the at least one vascular abnormality.

8. The method of claim 6 further comprising cooling a surface of the epidermal region of the target region of skin to prevent substantial unwanted injury to at least a portion of the epidermal region.

9. The method of claim 6 further comprising:
positioning each end within the target region of the skin at a first depth to treat the at least one vascular abnormality; and
repositioning each end within the target region of the skin at a second depth to treat the at least one pigmentary abnormality.

10. An apparatus for treating skin comprising:
a first plurality of waveguides, each first waveguide having a first end, the first plurality of waveguides adapted for penetrating an epidermis of the skin, positioning each first end at about a first depth within the skin, and delivering electromagnetic radiation through the first plurality of waveguides to form a plurality of first injuries about the first depth; and
a second plurality of waveguides, each second waveguide having a second end, the second plurality of waveguides adapted for penetrating the epidermis, positioning each second end at about a second depth within the skin, and delivering electromagnetic radiation through the second plurality of waveguides to form a plurality of second injuries about the second depth.

11. A method for treating skin comprising:
penetrating an epidermis of the skin with a first plurality of waveguides, each first waveguide having a first end, and a second plurality of waveguides, each second waveguide having a second end;
positioning each first end at about a first depth within the skin and each second end at about a second depth within the skin; and
delivering electromagnetic radiation through the first plurality of waveguides to form a plurality of first injuries about the first depth and delivering electromagnetic radiation through the second plurality of waveguides to form a plurality of second injuries about the second depth.

12. The method of claim 11 wherein the plurality of first injuries or the plurality of second injuries comprise a volume of necrotic thermal injury.

13. The method of claim 11 wherein the plurality of first injuries or the plurality of second injuries partially denature collagen to cause the skin to rejuvenate.

14. The method of claim 11 wherein the plurality of first injuries or the plurality of second injuries accelerate collagen synthesis in the skin to cause the skin to rejuvenate.

15. The method of claim 11 wherein the plurality of first injuries or the plurality of second injuries elicit a healing response that produces substantially unwrinkled skin.

16. The method of claim 11 wherein the plurality of first injuries or the plurality of second injuries activates fibroblasts which deposit increased amounts of extracellular matrix constituents in the skin.

17. The method of claim 11 wherein the plurality of first injuries or the plurality of second injuries are intervened by substantially undamaged skin.

18. The method of claim 11 further comprising forming a plurality of noncontiguous second injuries, disposed relative to the plurality of first injuries, to form a pattern of interspersed first injuries and second injuries.

19. The method of claim 11 wherein the plurality of first injuries are shallower than the plurality of second injuries.

20. The method of claim 11 wherein the electromagnetic radiation delivered to the first depth and the electromagnetic radiation delivered to the second depth differ in at least one parameter.

21. The method of claim 20 wherein the parameter includes at least one of fluence, wavelength, or pulse duration.

22. A method for treating skin comprising:
penetrating an epidermis of the skin with a plurality of waveguides, each waveguide having an end;
positioning each end at about a first depth within the skin;
delivering electromagnetic radiation through the plurality of waveguides to form a plurality of first injuries about the first depth;
positioning each end at about a second depth within the skin; and
delivering electromagnetic radiation through the second plurality of waveguides to form a plurality of second injuries about the second depth.
